(19) United States
(12) Patent Application Publication
(10) Pub. No.: US 2019/0275320 A1
(43) Pub. Date: Sep. 12, 2019

(54) SYSTEMS AND METHODS OF FACIAL TREATMENT AND STRAIN SENSING

(71) Applicant: Massachusetts Institute of Technology, Camdridge, MA (US)

(72) Inventors: Jeehwan Kim, Camdridge, MA (US); Kyusang Lee, Camdridge, MA (US); Yunjo Kim, Camdridge, MA (US); Kwangyong Jung, Camdridge, MA (US)

(73) Assignee: Massachusetts Institute of Technology, Camdridge, MA (US)

(21) Appl. No.: 16/348,128

(22) PCT Filed: Nov. 8, 2017
(86) PCT No.: PCT/US2017/060577

§ 371 Date: May 7, 2019

Related U.S. Application Data
(60) Provisional application No. 62/487,201, filed on Apr. 19, 2017.
(60) Provisional application No. 62/486,664, filed on Apr. 18, 2017.
(60) Provisional application No. 62/418,986, filed on Nov. 8, 2016.

(51) Int. Cl. A61N 1/32; A45D 44/00; A61N 1/30; A61B 5/00; A61N 1/04; A61N 1/06

(52) U.S. Cl. CPC A61N 1/325; A45D 44/002; A61N 1/30; A61B 5/442; A61B 2017/00057; A61N 1/06; A45D 2044/007; A45D 2200/205; A61N 1/0428

(57) ABSTRACT

A method of facial treatment of a user while wearing a treatment system is disclosed. The treatment system includes a flexible film and circuitry disposed on or within the flexible film. The method includes conformally disposing the flexible film over a face of the user and applying a radio frequency (RF) wave, generated by the circuitry, on skin of the face. The method eliminates the need for a user (or a third-party operator) to hold the device by hand. In addition, the thin film can be configured as a face mask allowing treatment over a large area of skin at any given time.

### CROSS-REFERENCES TO RELATED APPLICATIONS

This application claims the priority benefit under 35 U.S.C. § 119(e) of U.S. Application No. 62/418,986, filed Nov. 8, 2016, entitled “WEARABLE HIGH FREQUENCY DEVICE FOR SKIN CARE AND TRANSDERMAL DRUG DELIVERY,” U.S. Application No. 62/487,201, filed Apr. 19, 2017, entitled “OPTICAL STRAIN SENSORS FOR IN SITU SKIN TENSION MEASUREMENT,” and U.S. Application No. 62/486,664, filed Apr. 18, 2017, entitled “OPTICAL SKIN TENSION SENSORS,” each of which is hereby incorporated herein by reference in its entirety.

### BACKGROUND

High frequency radiation can be used for skin care applications, such as accelerating blood circulation, strengthening lymph activity, killing bacteria and viruses, and eliminating of acne and pimples. For example, in facial treatment, applying direct or indirect high frequency radiation over the face can reduce wrinkles, tighten skin, and improve skin texture and complexion. In another example, radio frequency (RF) skin tightening is an aesthetic technique that uses RF energy to heat tissue and stimulate subdermal collagen production in order to reduce the appearance of fine lines and loose skin.

However, conventional high-frequency skin care devices are usually bulky and inconvenient to use, and the treatment area is often localized by the active device size. For example, a typical high frequency facial treatment device includes a hand-held piece having a treatment tip whose size is usually on the order of centimeters or less. Therefore, the effect of the treatment, at any given moment, is localized within the area of the skin that is in contact with the tip. In addition, to perform treatment over the entire face, the user (or an additional operator) usually holds hand-held piece and moves it around the face, which can be inconvenient and time consuming.

### SUMMARY

Embodiments of the present invention include apparatus, systems, and methods for facial treatment and strain sensing. In one example, a method of using a treatment system is disclosed. The treatment system includes a flexible film and circuitry disposed on or within the flexible film. The method includes conformally disposing the flexible film over a face of a user and applying a radio frequency (RF) wave, generated by the circuitry, onto a skin of the face.

In another example, a wearable system for facial treatment of a user includes a flexible film made of a bio-compatible material and circuitry disposed on or within the flexible film and configured to generate an RF wave. When the flexible film is conformally disposed on a face of the user, the RF wave generated by the circuitry is applied to a skin of the face.

In yet another example, a method to estimate skin tension of a user includes disposing a periodic structure in conformal contact with a skin of the user and illuminating the periodic structure with a first light beam. The method also includes measuring a wavelength of a second light beam reflected, transmitted, and/or emitted by the periodic structure in response to the first light beam and estimating the skin tension of the user based at least in part on the wavelength of the second light beam.

In yet another example, a wearable system to estimate skin tension of a user includes a light source to emit a first light beam and a periodic structure, in optical communication with the light source and configured to be conformally attached to a skin of the user during use, to generate a second light beam in response to illumination by the first light beam. The system also includes a detector, in optical communication with the periodic structure, to measure a wavelength of the second light beam. The wavelength of the second light beam is indicative of the skin tension of the user.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIGS. 1A and 1B show schematics of a wearable system for high frequency facial treatment.

FIG. 2 shows a diagram of an RLC circuit that can be used in the wearable system shown in FIG. 1A.

FIG. 3 shows a schematic of an inductor that is fabricated via screening printing and can be used in the wearable system shown in FIG. 1A.

FIG. 4A is a photo of an RLC circuit made from screen printing technique.

FIGS. 4B and 4C are plots of impedance magnitude and phase, respectively, as a function of frequency for the circuit shown in FIG. 4A.

FIGS. 5A and 5B show schematics of a straight antenna and a meander antenna, respectively, that can be used for generating high frequency waves.

FIG. 6 shows a schematic of an emitter including antennas and a radio frequency (RF) choke to increase the efficiency of the antennas.

FIG. 7 shows measured S11 of the antennas shown in FIG. 6 with different amounts of bending to illustrate the flexibility of the antennas.

FIG. 8 illustrates a method of facial treatment using a wearable high frequency system.

FIGS. 9A-9D illustrate a method of fabricating a device including a distributed Bragg reflector (DBR) for optical strain sensing.

FIGS. 10A-10C illustrate a method of fabricating a device including a reflective grating for optical strain sensing.

FIGS. 11A and 11B illustrate optical strain sensing of skin using a distributed Bragg reflector.

FIGS. 12A and 12B illustrate optical strain sensing of skin using a reflective grating.

FIG. 13 illustrates a method of measuring local skin tension using a device including a DBR disposed on skin.

FIG. 14 illustrates a method of measuring local skin tension using a device including a grating disposed on skin.

FIGS. 15A and 15B show schematics of an optical strain sensor using a distributed fiber grating (DFG) fabricated in a fiber core.

FIG. 16 shows a schematic of a system to measure skin tension using a network of fibers including DFGs.

FIGS. 17A and 17B show schematics of an optical strain sensor using a photonic crystal band edge laser disposed on a flexible substrate.

FIGS. 18A-18C illustrate an optical strain sensor including a nano-antenna disposed within a stretchable substrate and a light emitting material disposed on the anno-antenna.

### DETAILED DESCRIPTION

Wearable Systems for High Frequency Facial Treatment

To address the inconvenience in conventional high frequency facial treatment techniques, systems and methods described herein integrated wave generation circuitry with a wearable and bio-compatible thin film that can be conformally attached to the face of a user. During operation, a user wears the thin film and the high frequency waves, such as radio frequency (RF) waves, generated by the circuitry, are applied over the user's face. In one example, a power source (e.g., a battery) can be integrated into the thin film to power the wave generation circuitry. In another example, the wave generation circuitry can be powered wirelessly via, for example, induction charging.

The high frequency wave generated by the circuitry can also be used to facilitate medicine delivery. For example, the thin film can be pre-loaded with medicine and the high frequency wave can facilitate driving the medicine into the skin of the user. In another example, the user can apply the medicine on the skin first and then wear the wearable system to drive the medicine into the skin.

The wearable approach described herein eliminates the need for a user (or a third-party operator) to hold the device and move it around the face for treatment. In addition, since the thin film can be configured as a face mask that covers substantially the entire face, the treatment can cover a large area at any given time, thereby addressing the issue of localized RF energy deposition in conventional devices. The combination with wireless energy transfer techniques further allows a user to conveniently control the operation of the circuitry and implement various types of treatment protocols. The wearable approach can also be combined with a conventional facial sheet mask to increase the efficiency of skin care or treatment.

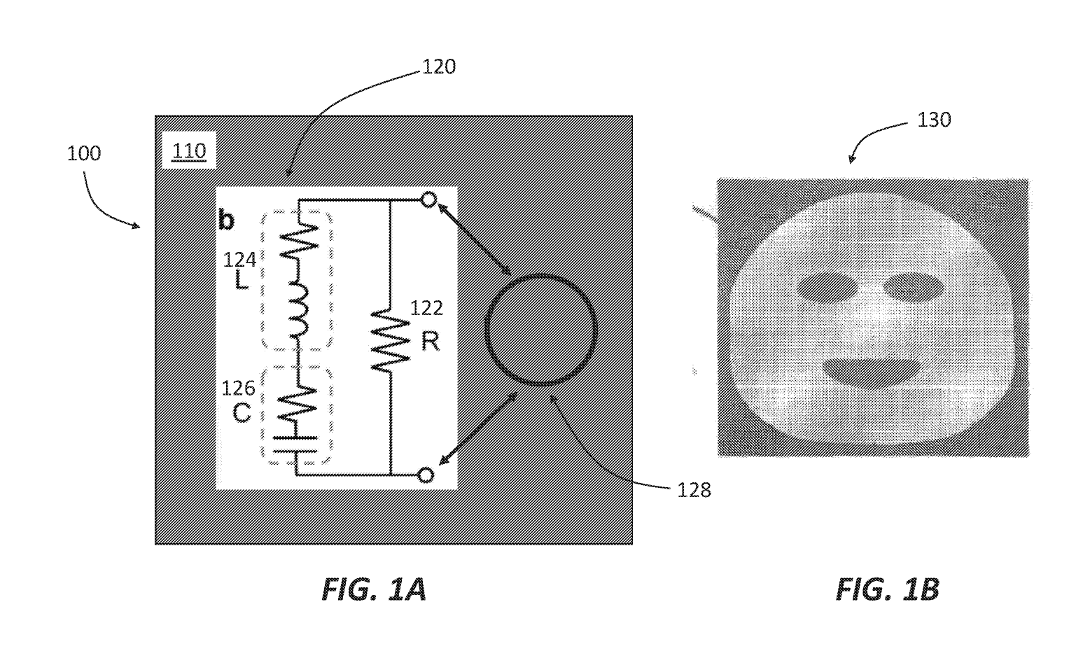

FIG. 1A shows a schematic of a wearable system 100 for high frequency facial treatment. The system 100 includes a thin film 110 and circuitry 120 disposed on or within the thin film 110. The circuitry 120, as illustrated in FIG. 1A, includes an RLC circuit configured to generate high frequency electromagnetic waves via RLC resonance. The RLC circuit includes a resistor 122, an inductor 124, and a capacitor 126. The inductor 124 and the capacitor 126 are connected in series, and the resistor 122 is connected in parallel with the inductor 124 and the capacitor 126. The system 100 also includes an antenna 130 operably coupled to the circuitry 120.

The circuitry 120 also includes an optional antenna 128 operably coupled to the RLC circuit. In one example, the antenna 128 includes a conductive ring configured to receive wireless energy from an external power source (e.g., via inductive charging) and the received energy is used to power the RLC circuit. In another example, the antenna 128 can be configured to emit high frequency electromagnetic waves for facial treatment. The antenna 128 can also be configured to receive control signals, from an external controller (not shown), to control the operation of the circuitry 120. For example, the control signal can control the radiation power of the high frequency waves. The thickness of the antenna 128 can be, for example, greater than the skin depth at the operation frequency of the circuitry 120.

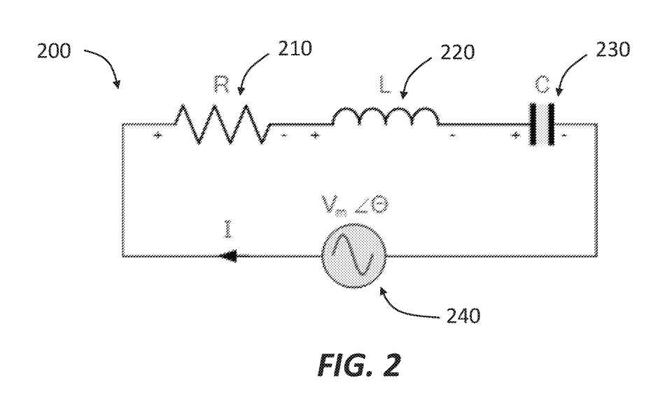

FIG. 2 shows a diagram of a series RLC circuit 200 that can be used in the wearable system 100 shown in FIG. 1A to generate high frequency waves. The circuit 200 includes a resistor 210, an inductor 220, and a capacitor 230 connected in series. A power supply 240 is employed to power the circuit 200. In one example, the power supply 240, such as a battery, can be integrated into the thin film. In another example, the power supply 240 can include an antenna to receive wireless power from an external source.

Various materials can be used to form the thin film 110. In general, the thin film 110 is bio-compatible, e.g., not harmful to the user's skin. For example, the thin film 110 can include silicone, polyethylene terephthalate (PET), poly(methyl methacrylate) (PMMA), or polydimethylsiloxane (PDMS). In some cases, the thin film 110 can also be sticky so as to facilitate the conformal contact with the user's face. Alternatively or additionally, system 100 can include an additional sticky interface (not shown in FIG. 1A) disposed on the thin film 110 and configured to be in contact with the user's face during use.

In some cases, the thin film 110 can be configured as a face mask (also referred to as a mask or mask sheet), as illustrated in FIG. 1B. In one example, the thin film 110 can include hydro gel, which is a material synthetically made by cross-linking hydrophilic molecules that can hold moisture. In this example, since hydro gel can be highly absorbent, serum or any other appropriate medicine can be applied in the thin film 110, which can prevent the serum from running off the thin film 110 or evaporating. The serum can be used to supplement the high frequency treatment.

In another example, the thin film 110 can include biocellulose, which is a fiber synthesized by specific bacteria. The fiber used herein can be very thin and therefore have good skin affinity. The fiber can also can hold up to 100 times of its dry weight in water (or serum).

In yet another example, the thin film 110 can include Tencel, which is an eco-friendly synthetic fiber obtained from, e.g., eucalyptus pulp. Tencel usually has extremely soft texture and can be very hypoallergenic. Accordingly, the thin film 110 made of Tencel can have good skin affinity and high air permeability, provides a very comfortable feeling for the user during facial treatment. In addition, the high permeability also allows quick heat dissipation when RF waves are used in the treatment.

In yet another example, the thin film 110 can include coconut gel that is made by specific bacteria during fermentation of coconut juice. Coconut gel can have a denser texture compared to hydrogel but maintain good skin affinity.

In yet another example, the thin film 110 can include cotton that is hypoallergenic and non-irritating. The cost of cotton is usually lower than that of other materials. Accordingly, a facial treatment system 100 made using cotton as the material for the thin film 110 can be a disposable treatment system.

The thickness of the thin film 110 can be substantially equal to or less than 50 μm (e.g., about 50 μm, about 45 μm, about 40 μm, about 35 μm, about 30 μm, about 25 μm, about 20 μm, or less, including any values and sub ranges in between). In use, the thin film 110 can be conformally applied over the face of the user. The conformal contact allows the thin film 110 to stay on the face of the user while the user is performing other tasks. In addition, the conformal contact also allows uniform irradiation of the face by the high frequency waves.

In one example, the circuitry 120 is disposed on the thin film 110. For example, the circuitry 120 can be fabricated on another substrate and then transferred to the thin film 110. Alternatively, the circuitry 120 can be directly fabricated on the thin film 110. In another example, the thin film 110 substantially encloses the circuitry 120. For example, the thin film 110 can include two layers disposed on opposite sides of the circuitry 120 so as to seal the circuitry 120. In one example, the two layers can be made of the same material. In another example, the two layers can be made of different materials. For example, the layer in contact with the face of the user can be made of a bio-compatible material (“first material”), while the other layer opposite the face of the user can be made of any other material (“second material”). The second material can be, for example, the original substrate (e.g., silicon, polyethylene terephthalate or PET) employed for fabricating the circuitry 120.

The frequency of the electromagnetic wave generated by the circuitry 120 can be for example, about 3 kHz to 300 MHz (e.g., about 3 kHz, about 5 kHz, about 10 kHz, about 20 kHz, about 30 kHz, about 50 kHz, about 100 kHz, about 200 kHz, about 300 kHz, about 500 kHz, about 1 MHz, about 2 MHz, about 3 MHz, about 5 MHz, about 10 MHz, about 20 MHz, about 30 MHz, about 50 MHz, about 100 MHz, about 200 MHz, or about 300 MHz, including any values and sub ranges in between). Different treatment protocols may use different frequencies. For example, skin cares may use frequencies from about 60 kHz to about 500 kHz. In another example, medical applications may use frequencies from about 3 kHz to about 300 MHz. When RLC circuit is used to generate the electromagnetic waves, the frequency ω₀ of the emitted wave can be determined by ω₀=1/(LC)^{1/2}, where L is the inductance of the inductor 124 and C is the capacitance of the capacitor 126.

Although FIG. 1A illustrates only one set of circuitry 120 for generating the high frequency waves, in practice, a single thin film 110 may host many circuits. For example, a single thin film 110 may support a circuitry array to more uniformly apply the high frequency waves over the face of the user. The circuits in this circuitry array can have different output powers, with circuitry closer to a target treatment area having a higher output power. For example, in high frequency treatment to reduce wrinkles, the circuitry disposed above the areas with more wrinkles (e.g., forehead or around the eyes,) can have a higher output power than circuitry disposed over other areas (e.g., the cheeks). Different circuits may also have different output frequencies so as to implement different protocols.

Compared to visible light or ultra-violet (UV) light treatment, RF treatment can penetrate deeper into the skin, thereby allowing deeper treatment or care of the skin. In some cases, the penetration depth of the high frequency waves generated by the circuitry 120 can be greater than 5 μm (e.g., about 5 μm, about 10 μm, about 15 μm, about 20 μm, about 25 μm, about 30 μm, or greater, including any values and sub ranges in between). In operation, the user can adjust the radiation power to control the penetration depth.

The wearable system 100 can also be configured for medicine delivery. For example, medicine can be pre-loaded into or onto the thin film 110 and the high frequency waves generated by the circuitry 120 can facilitate the delivery of the medicine into or onto the skin of the user. In this example, the wearable system 100 can function as a treatment patch. Alternatively or additionally, the user can apply the medicine on his or her face and then wear the system 100 to increase the delivery efficiency using the high frequency waves generated by the circuitry 120. FIGS. 1A and 1B use facial treatment as an example to illustrate the wearable system 100.

In practice, however, the wearable system 100 can be applied to any area of the skin. In addition, other than skin area and treatment, the wearable system 100 can also be used for other treatment. For example, the wearable system 100 may be used for arthritis treatment using its heat generation and drug delivery capabilities.

Methods of Fabricating Wearable Systems for High Frequency Treatment

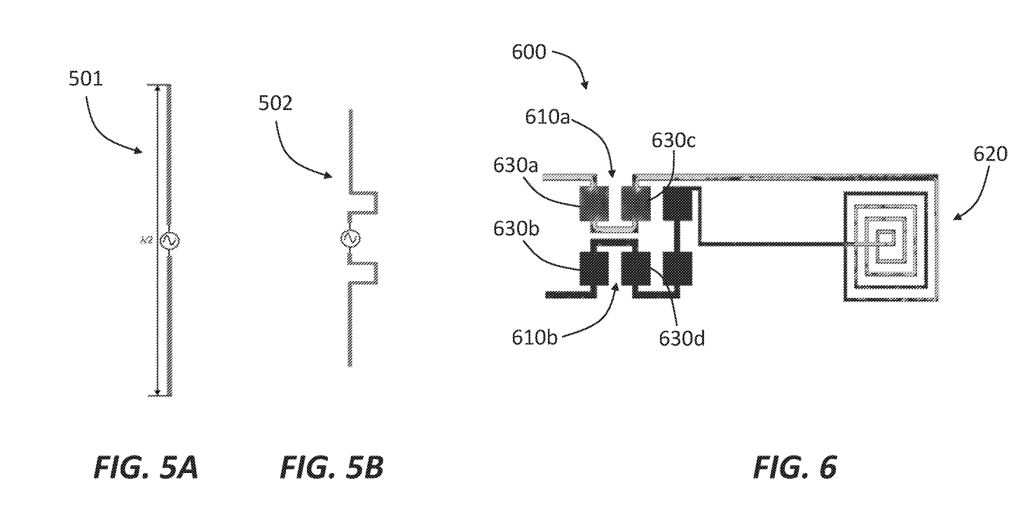

The wearable system 100 shown in FIG. 1A can be fabricated via various methods. In one example, the circuitry 120 can be fabricated on the thin film 110 via screen printing technique. In another example, electroplating can be used to manufacture the circuitry 120. In yet another example, the circuitry 120 can be fabricated via photolithography on a metal thin film. In yet another example, at least a portion of the circuitry 120 can be fabricated via vacuum thermal evaporation (see, e.g., FIGS. 5A and 5B).

Out of these methods, fabrication of electronic devices and circuits by additive printing processes (e.g., screen printing) has a number of advantages over printed circuit board (PCB)-based manufacturing techniques. For example, since many components of a circuit can be made of the same material (e.g., metal material for contacts and interconnects), printing allows multiple components to be fabricated simultaneously, thereby reducing the number of processing steps and the material cost. In addition, the low temperatures used in printing are compatible with flexible and inexpensive plastic substrates, allowing fabrication of large-area electronics using high-speed roll-to-roll manufacturing processes.

In some cases, the additive printing technique can be combined with surface-mount technology (SMT) to form a hybrid approach. In this hybrid approach, some electronic components are attached or mounted at low temperature onto the substrates alongside the printed components.

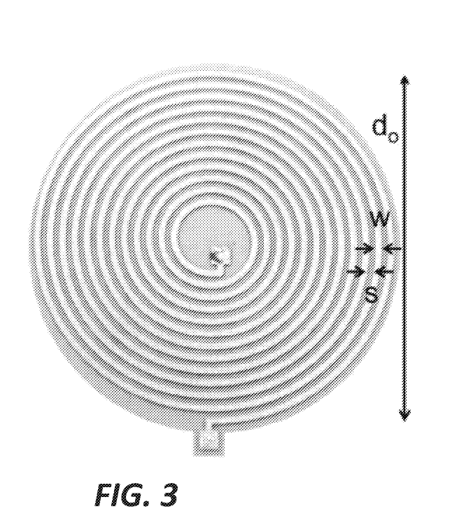

FIG. 3 shows a schematic of an inductor that can be fabricated via screen printing. The inductance and DC resistance of a range of inductor geometries can be calculated based on the current sheet model. In one example, the inductor can include a circular shape as shown in FIG. 3. In another example, the inductor can have a polygon geometry (see, e.g., 520 in FIG. 5).

The inductance and DC resistance of the inductor shown in FIG. 3 can be described by a few parameters: the outer diameter d₀, the turn width w (also referred to as the line width), the turn spacing s, the number of turns n, and the sheet resistance Rₛₕₑₑₜ of the conductor material forming the inductor. In FIG. 3, the inductor has 12 turns, i.e., n=12. Without being bound by any theory or particular mode of operation, the inductance L of the inductor shown in FIG. 3 can be calculated according to the current sheet model, in which:

$$L = \frac{μ {n}^{2} {d}_{avg}}{2} [ ln ( \frac{2.46}{ρ} ) + 0.2 {ρ}^{2} ] \qquad ( 1 )$$

where μ is the permeability of the core (in this case, air), d_{avg} is the average diameter d_{avg}=(dₒᵤₜ+dᵢₙ)/2, ρ is the fill ratio, i.e., ρ=(dₒᵤₜ−dᵢₙ)/(dₒᵤₜ+dᵢₙ), and dᵢₙ is the inner diameter: dᵢₙ=dₒᵤₜ−2n(w+s). The DC resistance can be calculated by: R_{dc}=Rₛₕₑₑₜl/w using the length l of the spiral, where l=πn[dᵢₙ+(w+s)(n−1)].

In practice, the outer diameter d₀ of the inductor can be, for example, from about 5 mm to about 30 cm (e.g., about 5 mm, about 1 cm, about 2 cm, about 3 cm, about 5 cm, about 10 cm, about 20 cm, about 25 cm, or about 30 cm, including any values and sub ranges in between). The turn width w (i.e., the width of the metal strip forming the inductor) can be, for example, from about 50 μm to about 10 mm (e.g., about 50 μm, about 100 μm, about 200 μm, about 300 μm, about 500 μm, about 1 mm, about 2 mm, about 3 mm, about 5 mm, or about 10 mm, including any values and sub ranges in between). The turn spacing s can be, for example, comparable to the turn width, i.e., about 50 μm to about 10 mm. The number of turns n can be, for example, greater than 5 (e.g., 5 turns, 10 turns, 15 turns, 20 turns, 30 turns, or more, including any values and sub ranges in between). In general, the inductance (and the resistance) of the inductor increases as the outer diameter do and number of turns n are increased, or as the turn width w is decreased.

Generally, it can be helpful for the inductor to have low DC resistance to reduce electrical losses. For the inductor shown in FIG. 3, a design and fabrication process to achieve a given inductance with minimum resistance can be as follows: first, the largest allowable outer diameter d₀ can be determined based on the spatial constraints imposed by the application. Then, the turn width w can be made as large as possible while still allowing the desired inductance to be reached, resulting in a high fill ratio. Reducing the sheet resistance of the metal material can further reduce the DC resistance without impacting the inductance. The sheet resistance of the metal material can be reduced by increasing the thickness or by using a material with higher conductivity.

The capacitor (e.g., 126 in FIG. 1A) used in the wearable system can include one or more dielectric layers disposed between two metal layers (i.e., electrodes). To reduce the footprint for a given capacitance, it can be helpful to use a capacitor with a large specific capacitance, which is equal to the dielectric permittivity E divided by the thickness of the dielectric layer. In one example, the dielectric layer can include a barium titanate (BaTiO₃) composite since its permittivity E is usually greater than that of other solution-processed organic dielectrics. The metal layer can include silver or another highly conductive material.

The thickness of dielectric layer in the capacitor can be, for example, about 2 μm to about 200 μm (e.g., about 2 μm, about 3 μm, about 5 μm, about 10 μm, about 20 μm, about 30 μm, about 50 μm, about 100 μm, about 150 μm, or about 200 μm, including any values and sub ranges in between). The area of the capacitor can be, for example, about 0.1 cm² to about 100 cm² (e.g., 0.1 cm², 0.2 cm², 0.3 cm², 0.5 cm², 1 cm², 2 cm², 3 cm², 5 cm², 10 cm², 20 cm², 30 cm², 50 cm², or 100 cm², including any values and sub ranges in between).

A number of approaches can be used to increase the capacitance. For example, a higher dielectric constant can increase the specific capacitance. This can be achieved by increasing the concentration of barium titanate particles in the ink. In another example, the thickness of the dielectric layer can be decreased to increase the capacitance. In yet another example, the capacitor can include multiple alternating layers of metal and dielectric.

The resistor (e.g., 122 in FIG. 1A) used in the wearable system can include one or more strips of conductive material. When multiple strips are used, the strips can be connected in parallel to decrease the resistance. The material of the resistor can be, for example, carbon, which is compatible with screen printing using carbon ink.

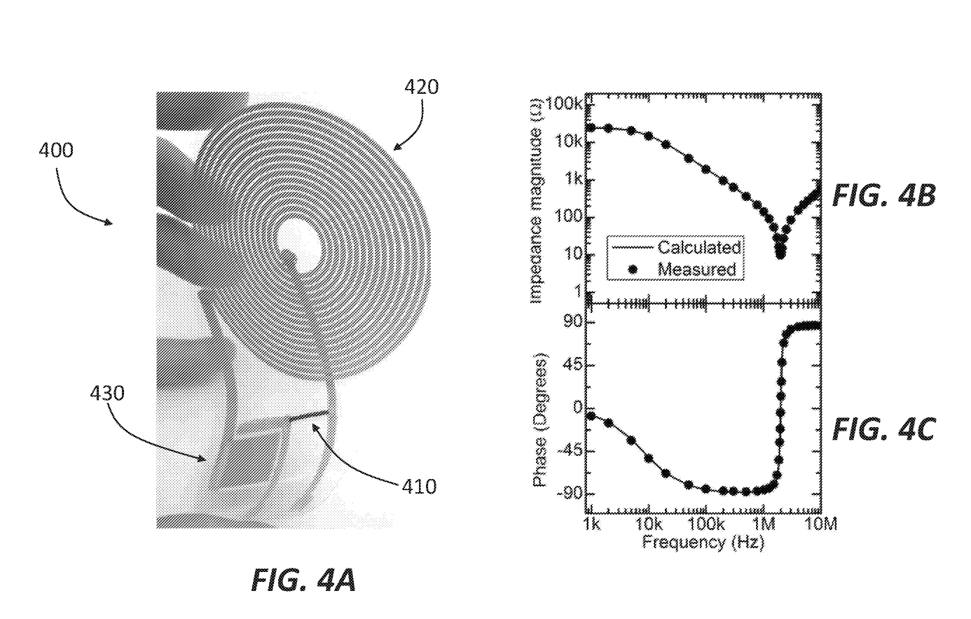

FIG. 4A is a photo of an RLC circuit 400 fabricated via screen printing technique. The circuit 400 includes a resistor 410, an inductor 420, and a capacitor 430. The inductor 420 and the capacitor 430 are connected in series, and the resistor 410 is connected in parallel with the combination of the inductor 420 and the capacitor 430. In the circuit 400, the inductance of the inductor 420 is about 8 μH, the capacitance of the capacitor 430 is about 0.8 nF, and the resistance of the resistor 410 is about 25 kΩ. The behavior of this series-parallel combination is dominated by each of the three components (resistor 410, inductor 420, and capacitor 430) at different frequencies, allowing the performance of each one to be highlighted and assessed.

FIGS. 4B and 4C are plots showing the impedance magnitude and phase, respectively, as a function of frequency for the circuit 400 shown in FIG. 4A. Both calculated and measured values are plotted in FIGS. 4B and 4C for comparison. At low frequency, the behavior of the circuit 400 is dominated by the 25 kΩ resistor 410. As the frequency increases, the impedance of the LC path decreases, and the overall circuit behavior is capacitive until the resonant frequency of 2.0 MHz. Above the resonant frequency, the inductor impedance dominates.

The circuit 400 shown in FIG. 4A can be fabricated as follows. The passive component layers can be screen printed onto flexible PET substrates (e.g., having a thickness of about 50 μm-80 μm) using an Asys ASP01M screen printer and stainless steel screens supplied by Dynamesh Inc. The mesh size can be, for example, 400 threads per inch for the metal layers and 250 threads per inch for the dielectric and resistor layers. Screen printing can be performed using a squeegee force of 55 N, print speed of 60 mm/s, snap-off distance of 1.5 mm, and Serilor squeegees with hardness of 65 durometer (for metal and resistor layers) or 75 durometer (for dielectric layer).

The ink for the conductive components (e.g., inductors and contacts in capacitors and resistors) can be, for example, silver micro-flake ink (e.g., Dupont 5082 or Dupont 5064H). The ink for the resistor can be carbon (e.g., Dupont 7082). For the capacitor dielectric, Conductive Compounds BT-101 barium titanate dielectric can be used. Each coat of dielectric can be produced using a double pass (wet-wet) print cycle to improve uniformity of the film.

In addition to screen printing, vacuum thermal evaporation can also be used to fabricate some components of the circuitry in a wearable system for high frequency treatment. FIGS. 5A and 5B show schematics of antennas that can be fabricated via vacuum thermal evaporation. FIG. 5A shows a straight λ/2 antenna 501, where A is the wavelength of the radiation that can be emitted by the antenna. FIG. 5B shows an antenna 502 having a meander configuration to reduce the footprint of the resulting circuitry. In operation, a thin metal film (e.g., Au film) can be deposited on a substrate and a shadow mask is then disposed on the metal film. The metal film is then patterned using vacuum thermal evaporation of Au through a shadow mask so as to form the antennas 501 and 502.

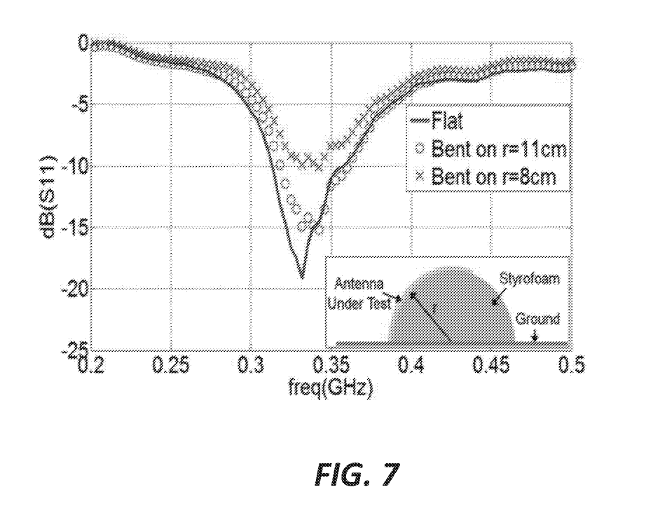

FIG. 6 shows a schematic of an emitter 600 including antennas 610a and 610b connected with a radio frequency (RF) choke 620. The RF choke 620 is basically an inductor, which can confine RF current into the antennas 610a and 610b and reduce RF current in other sections so as to increase the efficiency of the antennas 610a and 610b. The emitter 600 also includes several optional power sources 630a-630d, which can be, for example, solar cells.

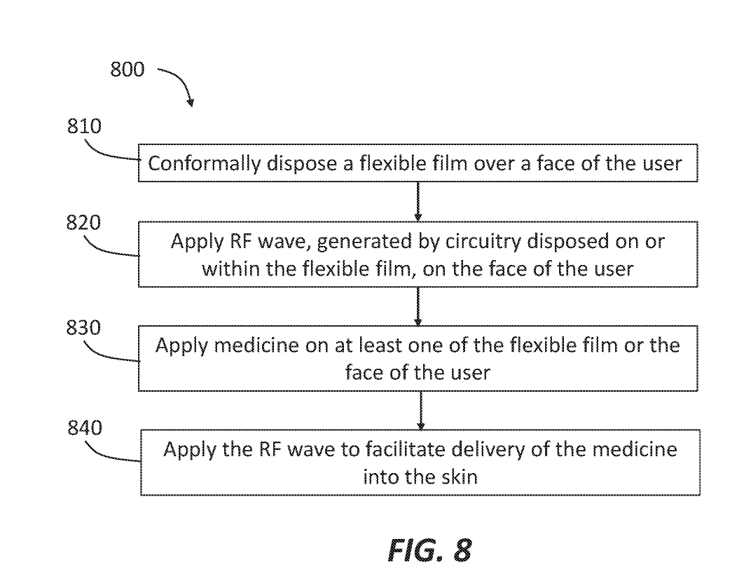

FIG. 7 shows the measured S11 (also referred to as return loss) of the emitter 600 shown in FIG. 6. The emitter 600 are conformally disposed on Styrofoam to achieve the bending. Radii of 11 cm and 8 cm of the two Styrofoam are used to bend the emitter 600 into different degrees. It can be seen from FIG. 7 that the bending does not noticeably affect the resonant frequency of the antennas 610a and 610b. Accordingly, the emitter 600 can be integrated with flexible thin films (e.g., thin film 110 in FIG. 1A) to form a wearable treatment system. In operation, the emitter 600 can also be conformally disposed on the skin, such as the face skin.

More information about screen printing of electronic components can be found in Aminy E. Ostfeld, et al., Screen printed passive components for flexible power electronics, Scientific Reports 5, Article number: 15959 (2015), and Jungsuek Oh, et al., Flexible Antenna Integrated with an Epitaxial Lift-Off Solar Cell Array for Flapping-Wing Robots, IEEE Transactions On Antennas and Propagation, Vol. 62, No. 8, August 2014, each of which is hereby incorporated by reference herein in its entirety.

Facial Treatment with a Wearable Treatment System

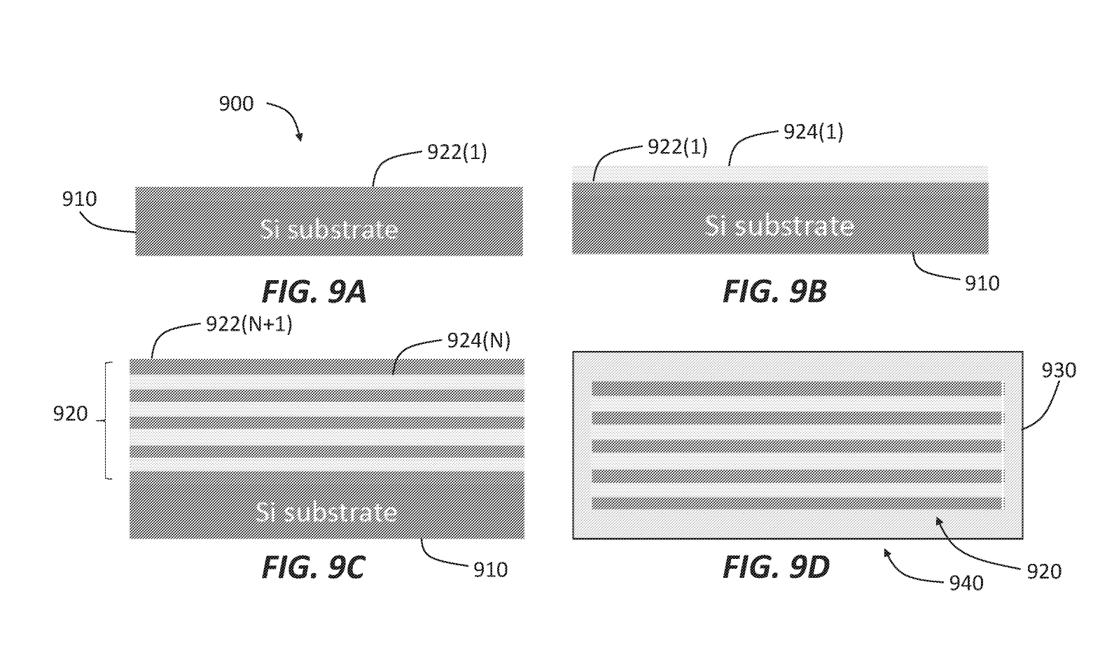

FIG. 8 illustrates a method 800 of facial treatment using a wearable treatment system that includes a flexible film and circuitry disposed on or within the flexible film. The method 800 includes conformally disposing the flexible film over a face of the user at 810 and applying a radio frequency (RF) wave, generated by the circuitry, to skin of the face at 820.

The thickness of the thin film can be substantially equal to or less than 50 μm so as to facilitate the conformal contact between the thin film and the face of the user. The material of the thin film can be any bio-compatible, such as silicone. The frequency of the RF wave can be, for example, about 3 kHz to about 300 MHz.

To generate the RF wave, the circuitry can include an RLC circuit powered by an antenna configured to receive wireless energy from an external source. The antenna can be further configured to receive control signals to operate the circuitry (e.g., controlling the power of the RF wave to be applied to the user). In some cases, the power of the RF wave can be configured to penetrate into the skin of the user for 10 μm or more.

The method 800 can also be used for medicine delivery. In this case, the method 800 can further include applying medicine over at least one of the face of the user or the flexible thin film, at 830, before conformally disposing the flexible film over the face of the user. The method 800 further includes applying the RF wave to facilitate delivering of the medicine into the skin of the user, at 840. In one example, the medicine is applied onto the face of the user. Alternatively or additionally, the medicine can be pre-loaded onto the thin film to form a treatment patch. The user can then wear the treatment patch and apply the RF wave generated by the circuitry to facilitate the delivery of the medicine into the skin.

Systems for Optical Strain Sensing

Cosmetology focuses significant efforts to promote lifestyle and products that can revitalize skin. One aspect of proper skin care and treatment is to reduce the tension in the skin to maintain a youthful complexion. While many cosmetic products offer to relieve skin tension, there are little experimental studies that can quantify the strain experienced by skin. In this application, system and methods employ an elastomeric optical nanostructure to sense skin tension by monitoring the light reflected, transmitted, or emitted by the nanostructure. The compression or tension of the skin can change the periodicity of the nanostructure, thereby changing the wavelength of the light reflected, transmitted, or emitted by the nanostructure.

This optical strain sensing approach has several advantages. First, it offers a quantifiable metric to gauge strain on skin by monitoring the change of wavelength using a spectrometer. In addition, the spatial resolution of the approach is fine and locally induced strain at micron scale can be detected using a 2-dimensional array of nanostructures with great lateral sensitivity. Accordingly, the skin tension distribution across the entire face or a portion of the face can be visually plotted. The optical nanostructure as described herein is flexible and can be conformally disposed on the face of the user. Therefore, this approach can function properly with uneven skin.

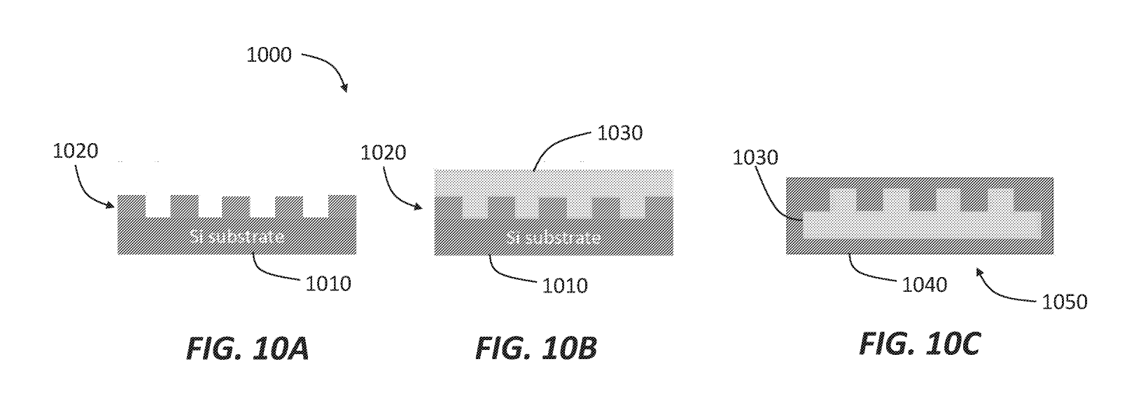

FIGS. 9A-9D illustrate a method 900 of fabricating a device for optical strain sensing. The method 900 includes disposing a first material 922(1) on a substrate 910 (e.g., a silicon substrate), as shown in FIG. 9A. In FIG. 9B, a second material 924(1) is disposed on the first material 922(1). The first material 922(1) has a first refractive index and the second material 924(1) has a second refractive index different from the first refractive index. The method 900 continues with the disposition of alternating layers to form a multilayer structure 920 as shown in FIG. 9C. The multilayer structure 920 can function as a distributed Bragg reflector (DBR), which can be configured to reflect light at a particular wavelength (e.g., in visible or near infrared region of electromagnetic spectrum) while passing through light at other wavelengths. The reflection wavelength of the multilayer structure 920 depends on its periodicity (or pitch). Therefore, when the periodicity of the multiplayer structure 920 changes (e.g., due to compression or stretching), the light reflected from the multilayer structure 920 can have a different wavelength. In FIG. 9D, the multilayer structure 920 is removed from the substrate 910 and encapsulated into an enclosure 930 to form a measurement device 940.

The multilayer structure 920 can use various types of materials. For example, the first material 922(1) can include a first elastomeric compound having a first refractive index and the second material 924(1) can include a second elastomeric compound having a second refractive index different from the first refractive index. The two elastomeric materials can be disposed via spin-coating. In another example, the multilayer structure 920 can include alternating layers of TiO₂ and SiO₂. In yet another example, the high- and low-refractive-index layers of the multilayer structure 920 can be deposited by oblique-angle deposition and include indium tin oxide (ITO) thin films with low and high porosities. In other words, the refractive index of the two materials 922(1) and 924(1) are adjusted by changing the porosity of the same material (i.e., ITO). The enclosure 930 can include an elastomeric resin and can be printed on both sides of the multilayer structure 920 so as to substantially seal the multilayer structure 920.

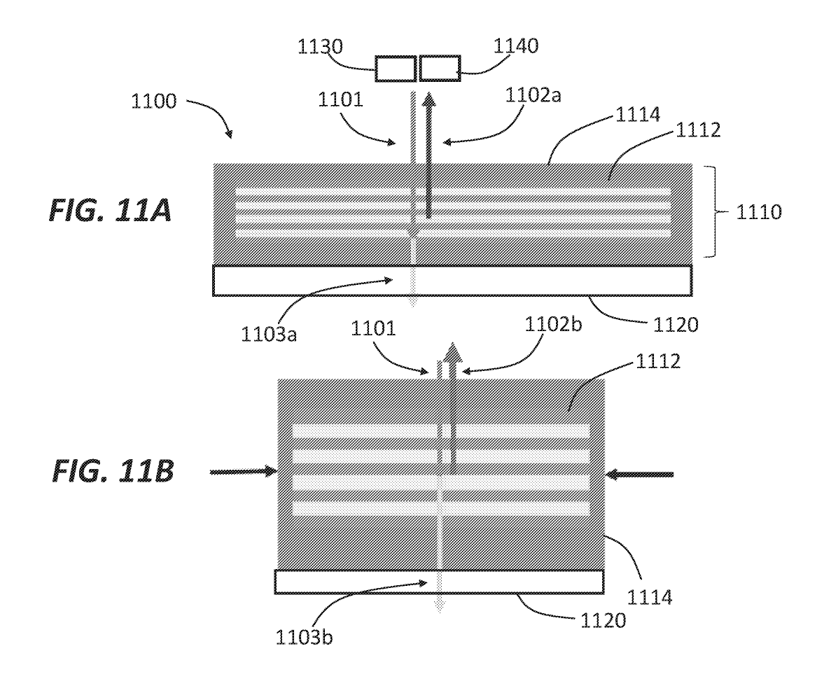

FIGS. 10A-10C illustrate a method 1000 of fabricating a device including a grating for optical strain sensing. The method 1000 includes forming a mask 1020 having a diffractive pattern on a substrate 1010 as shown in FIG. 10A. The mask 1020 can include, for example, SU-8 photoresist and can be patterned using ultra-violet (UV) light. In FIG. 10B, a grating 1030 is formed by depositing a material, such as polydimethylsiloxane (PDMS), onto the mask 1020. In FIG. 10C, the grating 1030 is removed from the mask 1020 and sealed in an enclosure 1040 so as to form a measurement device 1050.

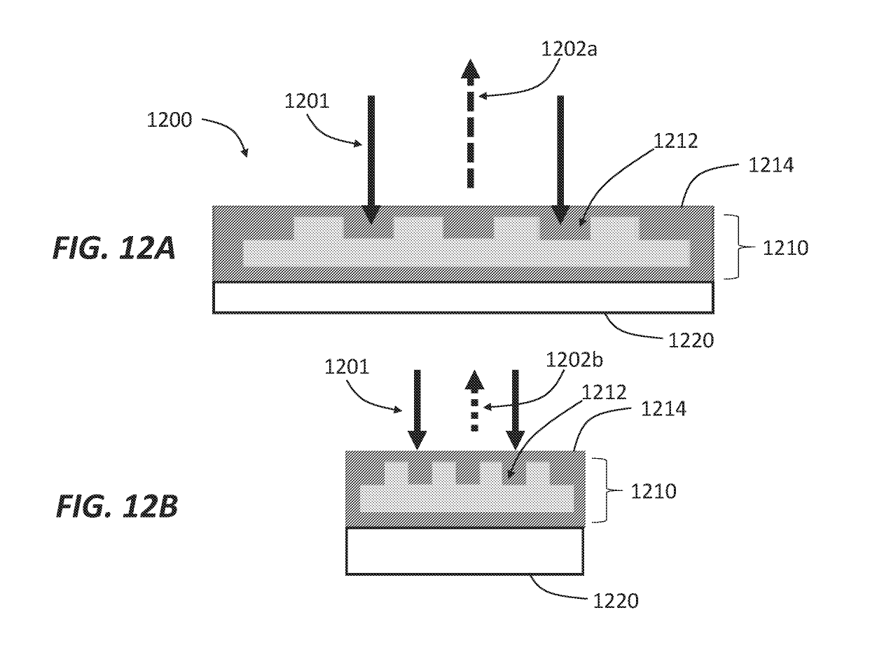

FIGS. 11A and 11B illustrate a method 1100 of measuring skin strain using a device 1110 that can be substantially similar to the device 940 shown in FIG. 9D. In this method, the device 1110 includes a DBR 1112 enclosed in an enclosure 1114 and is disposed on a patch of skin 1120. A light source 1130 is employed to deliver input light 1101 toward the device 1110 and a detector 1140 is employed to detect reflected light 1102a, while transmitted light 1103a passes through the device 1110. FIG. 11A illustrates that the skin 1120 is under strain (i.e., the skin 1120 is stretching), thereby stretching the device 1110. Under this stretching force, the DBR 1112 is also stretched due to its flexibility, thereby decreasing its periodicity. The decrease in the periodicity also decreases the wavelength of the reflected light 1102a, i.e., the reflected light 1102a is blue shifted.

In contrast, FIG. 11B illustrates that the skin 1120 is under tension (i.e., compression), thereby compressing the device 1110. Under this compressing force, the DBR 1112 is also compressed due to its flexibility, thereby increasing its periodicity. The increase in the periodicity also increases the wavelength of the reflected light 1102a, i.e., the reflected light 1102a is red shifted. FIGS. 11A and 11B illustrate that by monitoring the wavelength of the reflected light 1102a, the tension of the skin 1120 can be estimated.

The method 1000 can have very high sensitivity due to the micro- or nano-scale changes of the DBR 1112. In practice, the method 1000 can detect a change in skin tension of less than 1% (e.g., about 1%, about 0.8%, about 0.5%, about 0.3%, about 0.2%, about 0.1%, or less, including any values and sub ranges in between).

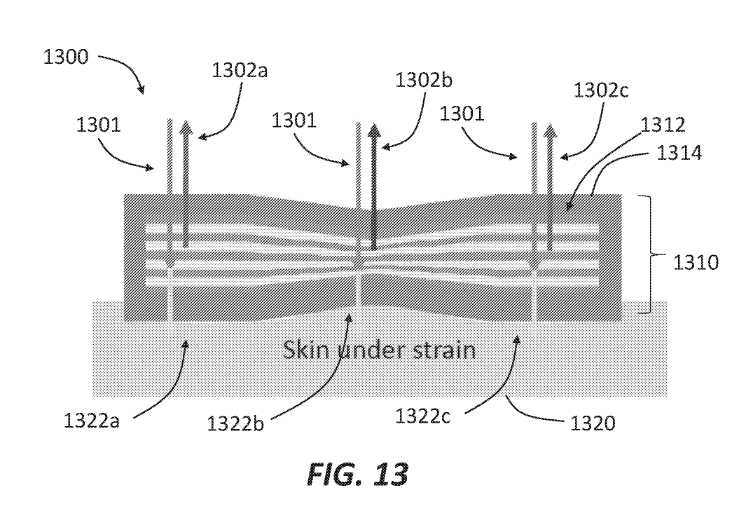

FIGS. 12A and 12B illustrate a method 1200 of measuring skin strain using a device 1210 that can be substantially similar to the device 1050 shown in FIG. 10C. In this method, the device 1210 includes a grating 1212 that is enclosed in an enclosure 1214 and disposed on a patch of skin 1220. Input light 1201 is delivered to illuminate the device 1210 and reflected light 1202a (in FIG. 12A) and 1202b (in FIG. 12B) are monitored to determine the tension of the skin 1220. FIG. 12A illustrates that the skin 1220 is under strain (i.e., stretching), thereby stretching the device 1210. Under this stretching force, the grating 1212 is also stretched and its periodicity increases accordingly. The increase in the periodicity also increases the wavelength of the reflected light 1202a, i.e., the reflected light 1202a is red shifted.

In contrast, FIG. 12B illustrates that the skin 1220 is under tension (i.e., compression), thereby compressing the device 1210. Under this compressing force, the grating 1212 is also compressed due to its flexibility, thereby decreasing its periodicity. The decrease in the periodicity also decreases the wavelength of the reflected light 1202a, i.e., the reflected light 1202a is blue shifted. FIGS. 12A and 12B illustrate that by monitoring the wavelength of the reflected light 1202a, the tension of the skin 1220 can be estimated.

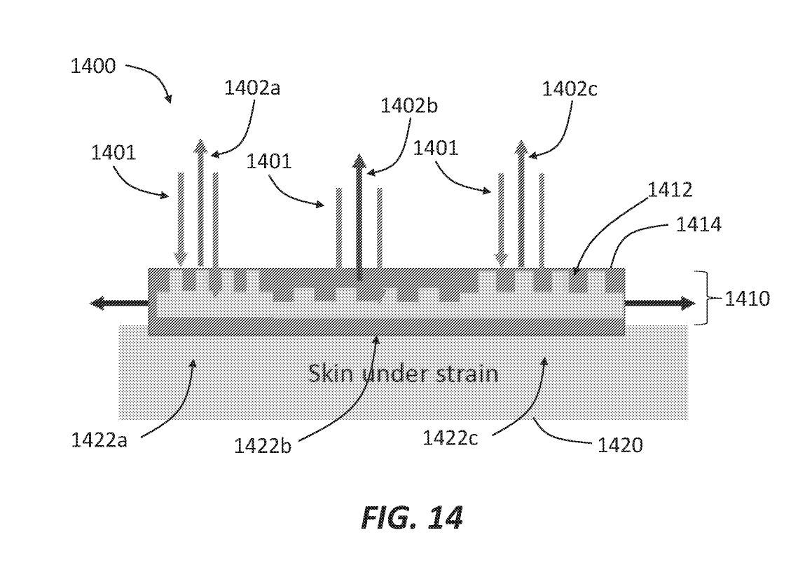

FIG. 13 illustrates a method 1300 of measuring local skin tension using a device 1310 disposed on a skin 1320. The device 1310 includes a DBR 1312 enclosed in an enclosure 1314. Input light 1301 shine on the device 1310 and reflected light beams 1302a-1302c are monitored to estimate the tension of the skin 1320. In FIG. 13, the skin 1320 has three areas 1322a, 1322b, and 1322c. The second area 1322b is under strain, while the other two areas 1322a and 1322b are under tension. In this case, the second reflected light beam 1302b is blue shifted and the other two reflected light beams 1302a and 1302c are red shifted. Accordingly, the tension distribution (or strain distribution) of the skin 1320 can be plotted.

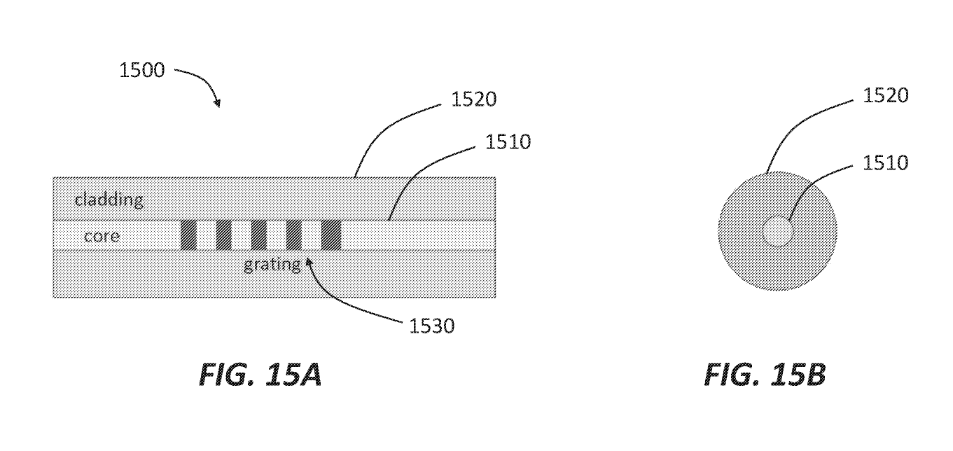

FIG. 14 illustrates a method 1400 of measuring local skin tension using a device 1410 disposed on a skin 1420. The device 1410 includes a grating 1412 enclosed in an enclosure 1414. Input light 1401 is delivered to the device 1410 and reflected light beams 1402a-1402c are monitored to estimate the tension of the skin 1420. In FIG. 14, the skin 1420 has three areas 1422a, 1422b, and 1422c. The first area 1422a is under tension, the second area 1422b is under strain, and the third area 1422c is in a neutral state (also referred to as a baseline state). Accordingly, the first reflected light beam 1402a is blue shifted, the second reflected light beam 1402b is red shifted, and the third reflected light beam 1402c does not change its wavelength. Accordingly, the tension distribution (or strain distribution) of the skin 1420 can be plotted.

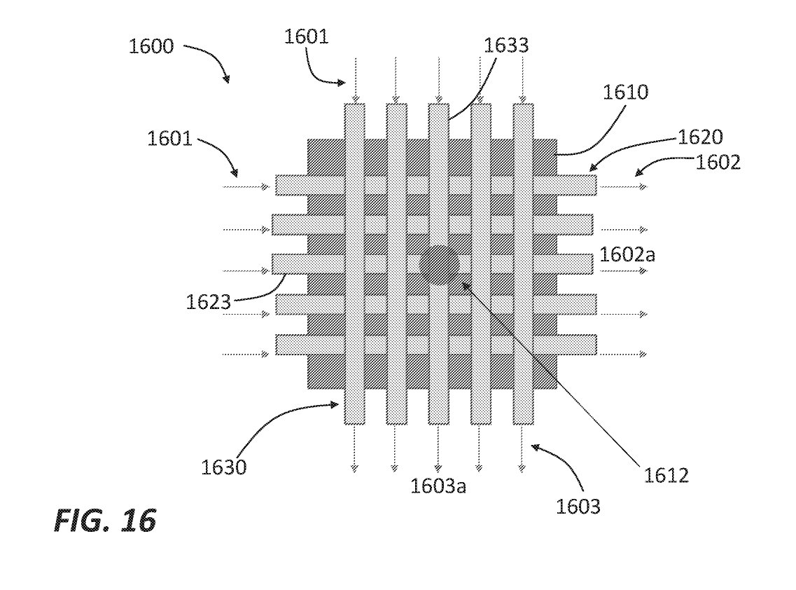

FIGS. 15A and 15B show schematics of an optical strain sensor 1500 using a distributed fiber grating (DFG) 1530 fabricated in a fiber core 1510 surrounded by a fiber cladding 1520. FIG. 15A shows a side view of the device 1500 and the FIG. 15B shows a cross sectional view of the device 1500. The DFG 1530 can be fabricated using the optical patterning technique. For example, the fiber core 1510 can include germanium-doped fiber material that is photosensitive. In other words, the refractive index of the fiber core 1510 changes under exposure to UV light. The amount of the change depends on the intensity and duration of the exposure. Therefore, the DFB 1530 can be formed by selective exposure of the fiber core 1510 to UV light.

In operation, the device 1500 is conformally disposed on the skin and input light is delivered to one end of the fiber core 1510 (e.g., via a coupler). As the skin strains or stretches, the DFG 1530 strains or stretches as well, thereby changing the periodicity of the DFG 1530. Accordingly, the wavelength of the light transmitted through the DFG 1530 also changes. More specifically, compression of the skin can blue shift the transmitted light, while stretching of the skin can red shift the transmitted light.

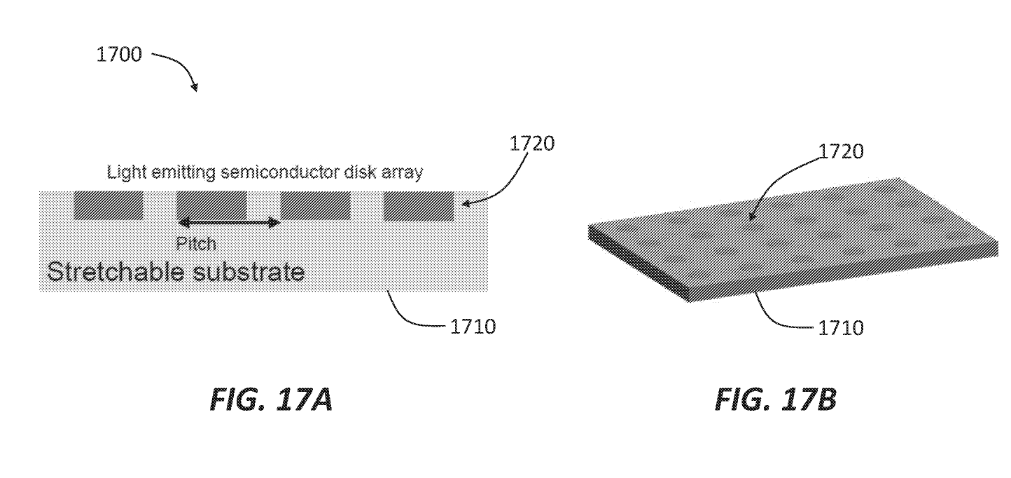

FIG. 16 shows a schematic of a system 1600 to measure skin tension using a network of fibers. The system 1600 includes a horizontal array of fibers 1620 and a vertical array of fibers 1630 disposed on a patch of skin 1610. Input light 1601 is delivered into each fiber and the corresponding transmitted light 1602 and 1603 are monitored. In one example, the light source providing the input light 1601 can be integrated with the fibers 1620 and 1630. In another example, the light source can be a separate unit and the input light 1601 can be coupled into the fibers 1620 and 1630 via directional couplers or any other appropriate couplers. Each fiber in the array of horizontal fibers 1620 and the array of vertical fibers 1630 includes a DFG (not shown in FIG. 16) in the core. The skin 1610 has a strain spot 1612, on which disposed a horizontal fiber 1623 and a vertical fiber 1633. Accordingly, the corresponding transmitted light beams 1602a and 1603a experience wavelength shifts. Accordingly, by observing the wavelength shifts in the light emitted from the fibers 1623 and 1633, the strain spot 1612 can be located as the cross section between the two fibers 1623 and 1633.

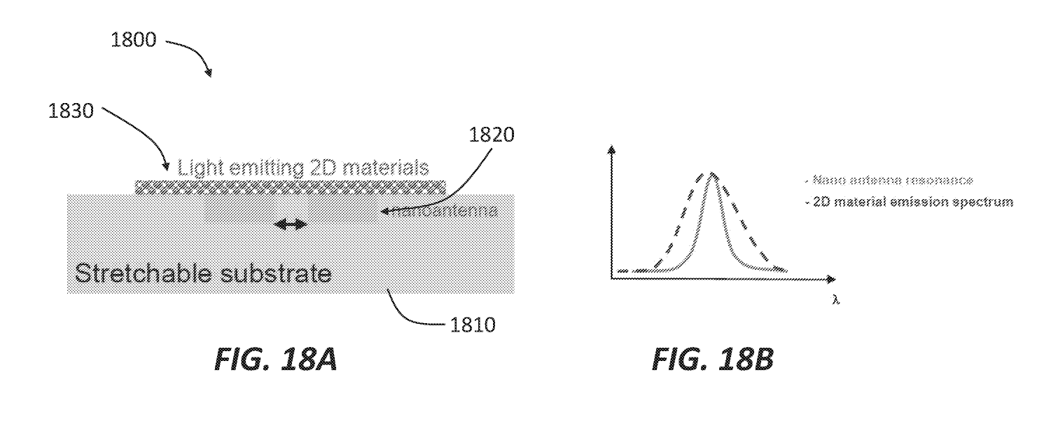

FIGS. 17A and 17B shows schematics of an optical strain sensor 1700 using a photonic crystal band edge laser 1720 disposed on a flexible substrate 1710. The photonic crystal band edge layer 1720 includes a two-dimensional (2D) array of light emitting semiconductor disks that are distributed in the flexible substrate 1720. In operation, the device is disposed on a user's skin. The emission wavelength of the laser 1720 depends on the pitch of the array, which in turn depends on the tension condition of the skin underneath the laser 1720. For example, the stretching of the skin also stretches the laser 1720, thereby increasing the pitch of the array and red shifting the emission wavelength. Conversely, the compression of the skin also compresses the laser 1720, thereby decreasing the pitch of the array and blue shifting the wavelength. Therefore, by monitoring the emission wavelength of the device 1700, the tension of the skin underneath the device 1700 can be estimated.

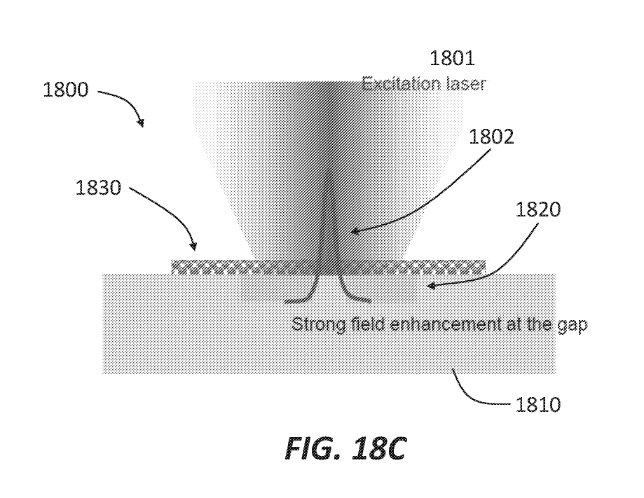

FIGS. 18A-18C illustrate an optical strain sensor 1800 including a nano-antenna 1820 disposed within a stretchable substrate 1810. A light emitting material 1830 is disposed on the nano-antenna 1820. In operation, the device 1800 is disposed on a user's skin and pump light 1801 (FIG. 1C) is employed to illuminate the light emitting material 1820 for optical excitation. The pump power can be, for example, about 20 mW or less (e.g., about 20 mW, about 15 mW, about 10 mW, about 8 mW, about 6 mW, about 4 mW, about 2 mW, about 1 mW, or less, including any values and sub ranges in between).

The wavelength of the emission light 1802 depends on the pitch of the nano-antenna 1820, which in turn depends on the stretching or tension of the underneath skin. For example, the compression of the skin can decrease the pitch of the nano-antenna 1820, thereby blue shifting the emission wavelength, while the stretching of the skin can increase the pitch of the nano-antenna 1820, thereby red shifting the emission wavelength.

In one example, the light emitting material 1820 includes a 2D material. In another example, the light emitting material 1820 includes a 3D material. The light emitting material 1820 can include, for example, transition metal dichalcogenide (TMD), which can be generally expressed as MX₂, where M is a transition metal atom (e.g., Mo, W, etc.) and X is a chalcogen atom (e.g., S, Se, or Te). Examples of TMD include MoS₂, WSe₂, and MoSe₂. In one example, the light emitting material 1820 can include a single layer of TMD. In another example, the light emitting material 1820 can include a heterostructure, such as a MoS₂/Silicon heterostructure or a WSe₂/MoS₂ heterostructure. In yet another example, the light emitting material 1820 can include quantum wells, which can include one semiconductor material (e.g., gallium arsenide) sandwiched between two layers of a material having a wider bandgap (e.g., aluminium arsenide). In another example, the quantum well can include indium gallium nitride sandwiched between two layers of gallium nitride.

The systems and methods illustrated in FIG. 9A-18C can be used in many applications. For example, these systems and methods can be employed to evaluate the effects of skin care or treatment. In general, a user can measure the skin tension before skin care or treatment and then measure again the skin tension after the skin care or treatment. The change of skin tension can be used to quantify the effect of the skin care or treatment.

For example, facial rejuvenation is a procedure to restore a youthful appearance to the human face. In one example, facial rejuvenation can be performed via surgical procedures (also referred to as invasive procedures), such as a brow lift (forehead lift), eye lift (blepharoplasty), facelift (rhytidectomy), chin lift, and neck lift. In another example, facial rejuvenation can be performed with non-surgical procedures, such as chemical peels, neuromodulator (e.g., injection of botox), dermal fillers, laser resurfacing, photo-rejuvenation, radiofrequency, and ultrasound. A user (or the service provider) can measure the skin tension before and after each facial rejuvenation to evaluate the effect of the treatment. The evaluation can then be used to instruct subsequent treatment.

In another example, these systems and methods can be employed to measure the strain on one area of the skin while the medicine or treatment is applied on another area of the skin. For example, certain antioxidant product can have beneficial effects on the entire skin system and the skin tension can be monitored at locations most convenient for measurement (e.g., face, arm, hand, etc.) to evaluate the efficacy of the product.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method of using a treatment system comprising a flexible film and circuitry disposed on or within the flexible film, the method comprising:
conformally disposing the flexible film over a face of a user; and
applying a radio frequency (RF) wave, generated by the circuitry, onto a skin of the face.

2. The method of claim 1, wherein the flexible film has a thickness substantially equal to or less than 50 μm.

3. The method of claim 1, wherein the flexible film comprises silicone.

4. The method of claim 1, wherein applying the RF wave comprises applying the RF wave at a frequency of about 3 kHz to about 300 MHz.

5. The method of claim 1, wherein applying the RF wave comprises delivering the RF wave into the skin of the face at a penetration depth substantially equal to or greater than 10 μm.

6. The method of claim 1, further comprising:
generating the RF wave using an RLC circuit in the circuitry; and
powering the RLC circuit via an antenna in the circuitry.

7. The method of claim 1, further comprising:
applying medicine to at least a portion of the face of the user before conformally disposing the flexible film over the face of the user; and
wherein applying the RF wave facilitates penetration of the medicine into the skin of the user.

8. The method of claim 1, further comprising:
applying medicine on the flexible film before conformally disposing the flexible film over the face of the user; and
wherein applying the RF wave facilitates penetration of the medicine into the skin of the user.

9. A wearable system for facial treatment of a user, the system comprising:
a flexible film comprising a bio-compatible material; and
circuitry disposed on or within the flexible film and configured to generate an RF wave,
wherein, when the flexible film is conformally disposed on a face of the user, the RF wave generated by the circuitry is applied to a skin of the face.

10. The wearable system of claim 9, wherein the flexible film has a thickness substantially equal to or less than 50 μm.

11. The wearable system of claim 9, wherein the bio-compatible material comprises silicone.

12. The wearable system of claim 9, wherein the circuitry comprises:
an RLC circuit to generate the RF wave; and
an antenna, in electrical communication with the RLC circuit, to receive power from an external source and power the RLC circuit.

13. The wearable system of claim 9, further comprising:
medicine disposed on the flexible film, wherein the circuitry is configured to facilitate penetration of the medicine into the skin of the user with the RF wave.

14. A method to estimate skin tension of a user, the method comprising:
disposing a periodic structure in conformal contact with a skin of the user;
illuminating the periodic structure with a first light beam;
measuring a wavelength of a second light beam reflected, transmitted, and/or emitted by the periodic structure in response to the first light beam; and
estimating the skin tension of the user based at least in part on the wavelength of the second light beam.

15. The method of claim 14, wherein:
disposing the periodic structure comprises orienting an optical axis of a distributed Bragg reflector (DBR) substantially perpendicular to the skin of the user, and
detecting the second light beam comprises detecting the second light beam reflected by the DBR.

16. The method of claim 14, wherein:
disposing the periodic structure comprises disposing a grating having a periodicity along a first direction on the skin of the user such that the first direction is substantially parallel to the skin of the user, and
detecting the second light beam comprises detecting the second light beam reflected by the grating.

17. The method of claim 14, wherein:
disposing the periodic structure comprises orienting an optical axis of a distributed fiber grating (DFG) substantially parallel to the skin of the user, and
detecting the second light beam comprises detecting the second light beam transmitted through the DFG.

18. The method of claim 14, wherein:
disposing the periodic structure comprises disposing a photonic crystal on the skin of the user, the periodic stricture comprising a light emitting material disposed in optical communication with the photonic crystal,
illuminating the periodic structure comprises optically exciting the light emitting material with the first light beam, and
detecting the second light beam comprises detecting the second light beam emitted by the light emitting material.

19. The method of claim 14, wherein the skin tension of the user is a first skin tension and the wavelength of the second light beam is a first wavelength, and the method further comprises:
removing the periodic structure from the skin of the user;
performing a skin treatment on the skin of the user;
disposing the periodic structure in conformal contact with the skin of the user after the skin treatment;
illuminating the periodic structure with a third light beam;
measuring a second wavelength of a fourth light beam reflected, transmitted, and/or emitted by the periodic structure;
estimating a second skin tension of the user based at least in part on the second wavelength of the fourth light beam; and
evaluating an efficacy of the skin treatment based at least in part on the first skin tension and the second skin tension.

20. A wearable system to estimate skin tension of a user, the system comprising:
a light source to emit a first light beam;
a periodic structure, in optical communication with the light source and configured to be conformally attached to a skin of the user during use, to generate a second light beam in response to illumination by the first light beam; and
a detector, in optical communication with the periodic structure, to measure a wavelength of the second light beam, the wavelength of the second light beam being indicative of the skin tension of the user.

21. The wearable system of claim 20, wherein the periodic structure comprises a distributed Bragg reflector (DBR) having an optical axis substantially perpendicular to the skin of the user during use and the periodic structure is configured to generate the second light beam via reflection of the first light beam by the DBR.

22. The wearable system of claim 20, wherein the periodic structure comprises a grating having a periodicity along a first direction substantially parallel to the skin of the user during use and the grating is configured to generate the second light beam via reflection of the first light beam.

23. The wearable system of claim 20, wherein the periodic structure comprises a distributed fiber grating (DFG) having an optical axis substantially parallel to the skin of the user during use and the DFG is configured to generate the second light beam via transmission of the first light beam.

24. The wearable system of claim 20, wherein the periodic structure comprises:
a photonic crystal; and
a light emitting material disposed in optical communication with the photonic crystal,
wherein the second light beam is emitted by the light emitting material in response to illumination by the first light beam.

25. The wearable system of claim 24, wherein the light emitting material comprises a two-dimensional (2D) material.

26. The wearable system of claim 20, further comprising:
an enclosure substantially enclosing the periodic structure, the enclosure comprising an elastomeric resin.
